(19) United States
(12) Patent Application Publication
(10) Pub. No.: US 2024/0350496 A1
(43) Pub. Date: Oct. 24, 2024

(54) Design, Synthesis, and Structure-Activity Relationships of Substituted Pyrido[3,2-d]Pyrimidines as Microtubule Targeting Agents and Anti-Cancer Agents

(71) Applicant: Duquesne University of The Holy Spirit, Pittsburgh, PA (US)

(72) Inventor: Aleem Gangjee, Allison Park, PA (US)

(21) Appl. No.: 18/638,213

(22) Filed: Apr. 17, 2024

Related U.S. Application Data
(60) Provisional application No. 63/496,728, filed on Apr. 18, 2023.

(51) Int. Cl. A61K 31/519; A61P 35/00; C07D 471/04

(52) U.S. Cl. CPC A61K 31/519; A61P 35/00; C07D 471/04

(57) ABSTRACT

A compound is provided having a structure of the formula:

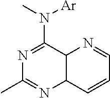

wherein Ar is a structure of the formula including at least one of 2, 3, 4, 6, 7, 8, or 9:

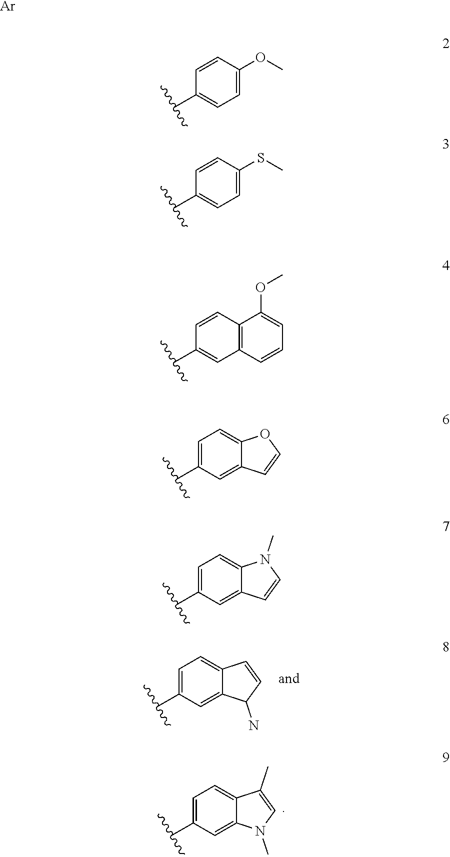

A compound having the formula

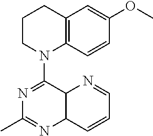

is also provided.

Compositions are provided that include one or more of these compounds and a pharmaceutically acceptable vehicle. A method of treating a patient having cancer is provided that includes administering to said patient a therapeutically effective amount of one or more of these compounds or these compositions for treating said patient.

### CROSS-REFERENCE TO RELATED APPLICATION

This utility non-provisional patent application claims the benefit of priority to U.S. Provisional Patent Application Ser. No. 63/496,728, filed Apr. 18, 2023. The entire contents of U.S. Provisional Patent Application Ser. No. 63/496,728 are incorporated by reference into this utility non-provisional patent application as if fully rewritten herein.

### STATEMENT REGARDING FEDERALLY SPONSORED RESEARCH OR DEVELOPMENT

Not Applicable,

### BACKGROUND OF THE INVENTION

### 1. Field of the Invention

This invention provides novel substituted pyrido[3,2-d]pyrimidines for use as microtubule targeting agents and anti-cancer agents.

### 2. Background Art

Cancer is the second leading cause of death worldwide, projected to account for over 1.9 million new cancer cases and approximately 609,820 deaths in the United States in 2023 alone.¹ Current major treatments for cancer management include surgery, chemotherapy, targeted therapy, radiation therapy, endocrine therapy, immunotherapy and gene therapy. Despite many advances in cancer chemotherapy, many cancers that initially respond to therapies eventually show development of resistance to the drugs. When these drugs stop working, patients often have few, if any, effective treatment options. This necessitates the development of novel small molecule chemotherapy agents to circumvent resistance mechanisms and improve disease outcome in patients.

Microtubules (MTs) are filamentous protein polymers composed of αβ-tubulin heterodimers,² and they form an extensive network within cells. MTs are involved in essential cellular processes, including mitosis, intracellular trafficking of proteins and organelles, cell-cell signaling and cellular migration. These roles are possible by the dynamic nature of MTs.^{2,3} MT targeting agents (MTAs) disturb αβ-tubulin heterodimer addition and loss from the MT, and this disrupts MT-dependent cellular processes and interferes with the proliferative signaling process, one of the hallmarks of cancer.²⁻⁶

There are two major classes of MTAs: 1) microtubule polymerizing agents bind to the taxol site (paclitaxel, docetaxel) or the laulimalide/peloruside A site on the microtubule and 2) microtubule depolymerizing agents bind to either the vinca domain (vincristine, vinblastine), the maytansine site or the colchicine site (combretastatin A4 (CA4) and verubulin).⁷⁻¹⁴ MTAs are one of the most successful class of cancer chemotherapeutics and the only agents effective against cancer cell lines with p53 mutations (39 cancer cell lines in the NCI 60-cell line panel).¹⁵ However, the clinical efficacy of MTAs such as paclitaxel is limited by multidrug resistance due to expression of the P-glycoprotein (Pgp) drug efflux pump.¹⁶⁻¹⁹ Fortunately, most MTAs that bind within the colchicine site circumvent Pgp mediated resistance.²⁰ Also, Colchicine site binding inhibitors (CSBIs) frequently act as vascular disrupting agents, inhibiting tumor growth by rapid shutdown of tumor blood flow.²¹⁻²³ CA4 is a cis-stilbene that binds to the colchicine site and its polyphosphorylated analog combretastatin A-4 phosphate (CA4-P) is currently in clinical trials.^{20,21} However, there are no approved CSBIs for the treatment of cancer, and this suggests the importance of developing colchicine site agents with effectiveness against cell lines expressing multidrug resistance which could have potential advantages in patients who fail to respond to current MTAs, such as paclitaxel.

### SUMMARY OF THE INVENTION

In certain embodiments of this invention, 2-methylpyrido[3,2-d]pyrimidine compound 2 is provided, using Verubulin²²⁻²³ (quinazoline scaffold) and known in the art compound 1²⁴ (pyrido[3,2-d]pyrimidine scaffold) as our leads, and compounds 3-9 of this invention are provided with different substitutions at the N4-position of compound 2 of this invention.

In certain embodiments of this invention, a compound is provided having a structure of the formula:

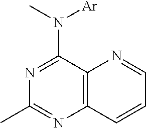

wherein Ar is a structure of the formula selected from one of the group consisting of 2, 3, 4, 6, 7, 8, and 9:

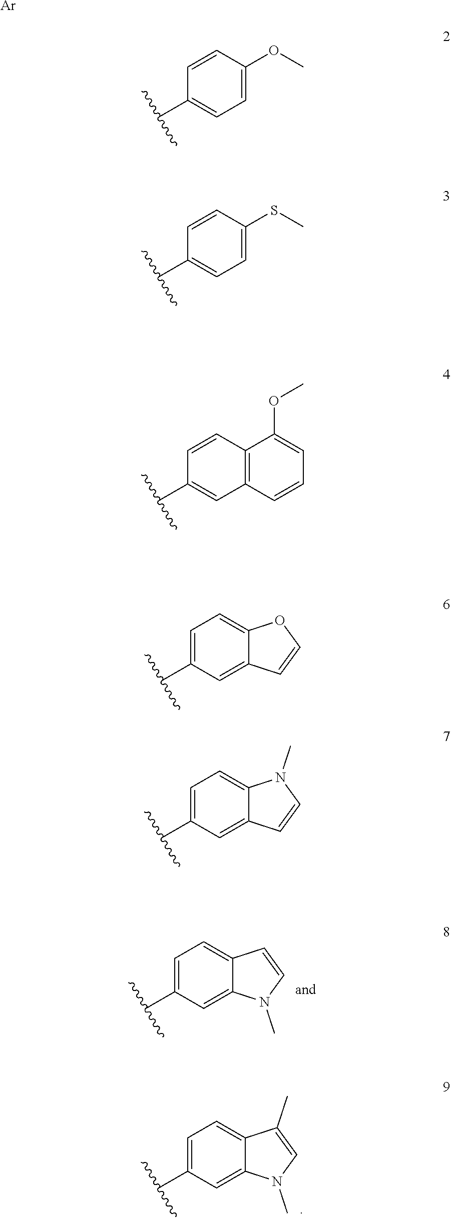

In another embodiment of this invention, a composition is provided comprising a compound having a structure of the formula:

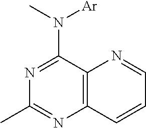

wherein Ar is a structure of the formula selected from one of the group consisting of 2, 3, 4, 6, 7, 8, and 9:

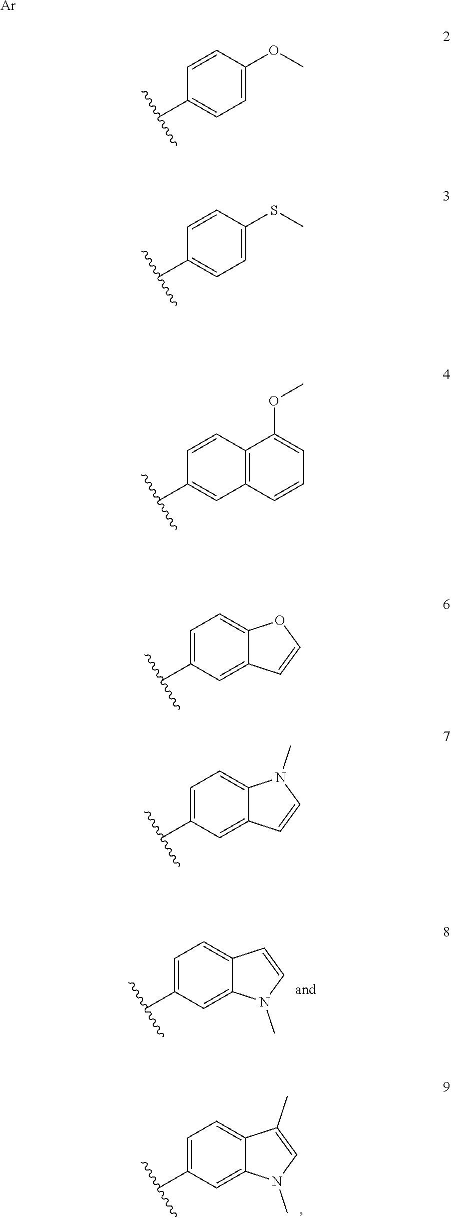

and a pharmaceutically acceptable vehicle.

In another embodiment of this invention, a method of treating a patient having cancer is provided comprising administering to said patient a therapeutically effective amount of a compound having a structure of the formula:

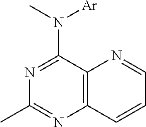

wherein Ar is a structure of the formula selected from one of the group consisting of 2, 3, 4, 6, 7, 8, and 9:

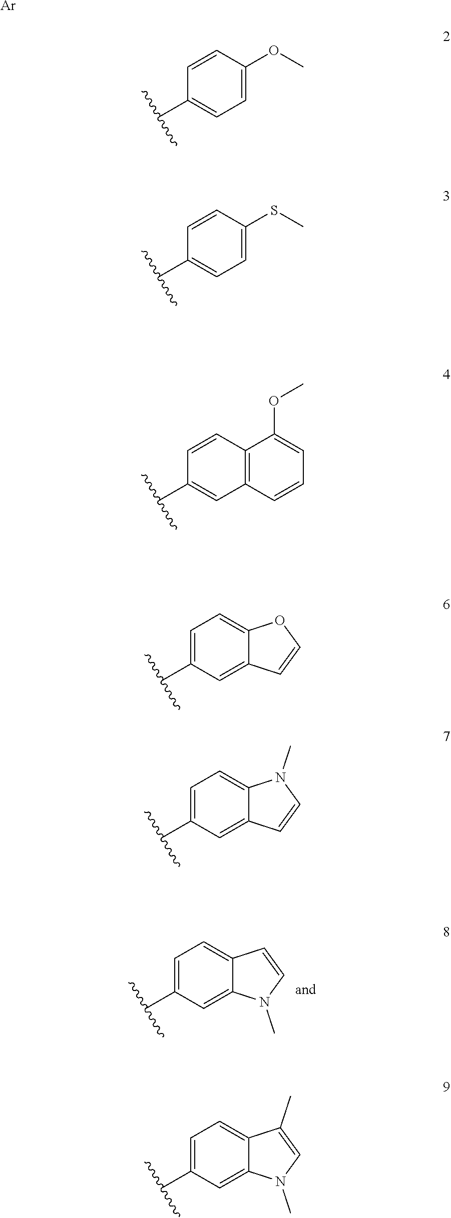

for treating said patient.

In another embodiment of this invention, a method of treating a patient having cancer is provided comprising administering to said patient a therapeutically effective amount of a composition comprising a compound having a structure of the formula:

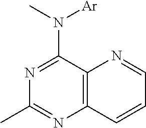

wherein Ar is a structure of the formula selected from one of the group consisting of 2, 3, 4, 6, 7, 8, and 9:

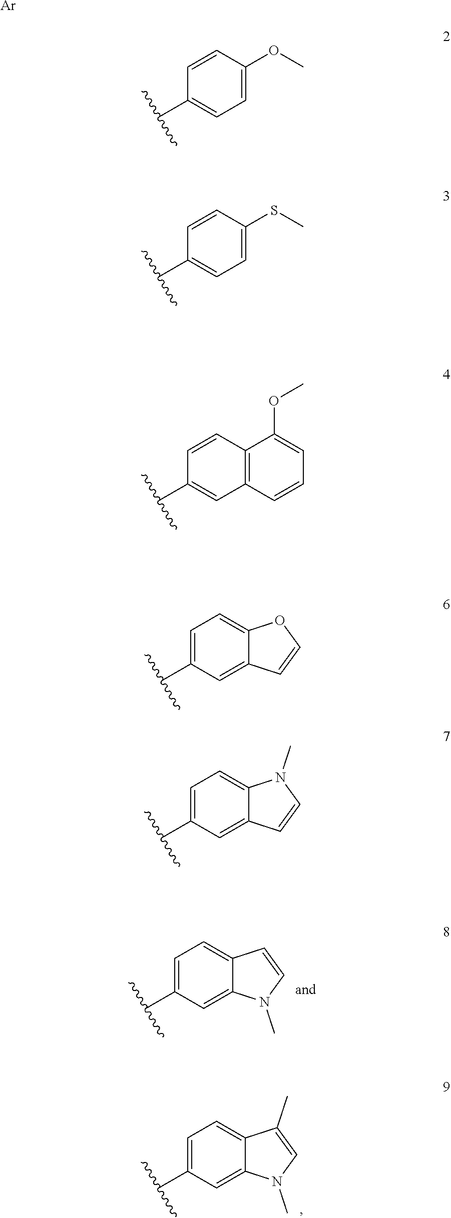

and a pharmaceutically acceptable vehicle, for treating said patient.

In another embodiment of this invention, a compound having the structure of the formula:

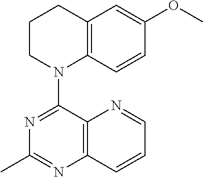

is provided.

In another embodiment of this invention, a composition is provided comprising a compound having a structure of the formula:

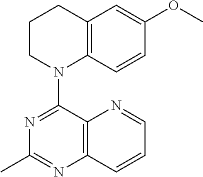

and a pharmaceutically acceptable vehicle.

Another embodiment of this invention provides a method of treating a patient having cancer comprising administering to said patient a therapeutically effective amount of a compound having a structure of the formula:

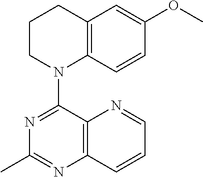

for treating said patient.

In another embodiment of this invention, a method of treating a patient having cancer is provided comprising administering to said patient a therapeutically effective amount of a composition comprising a compound having a structure of the formula:

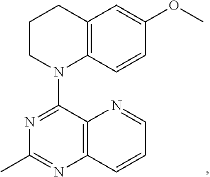

and a pharmaceutically acceptable vehicle, for treating said patient.

### BRIEF DESCRIPTION OF THE DRAWINGS

A full description of the invention may be gained from the following description of the embodiments of the invention when read in conjunction with the accompanying drawings:

FIG. 1 shows the structures of known in the art microtubule targeting agents.

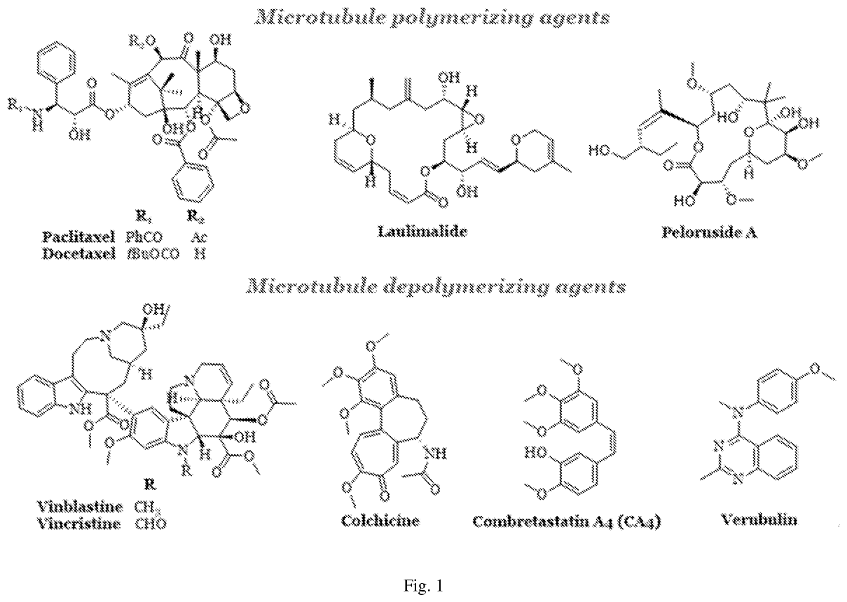

FIG. 2 shows the structure of a known in the art lead compound 1.

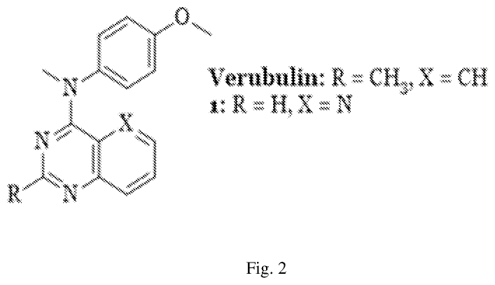

FIG. 3 shows the structures of compounds 2-9 of this invention.

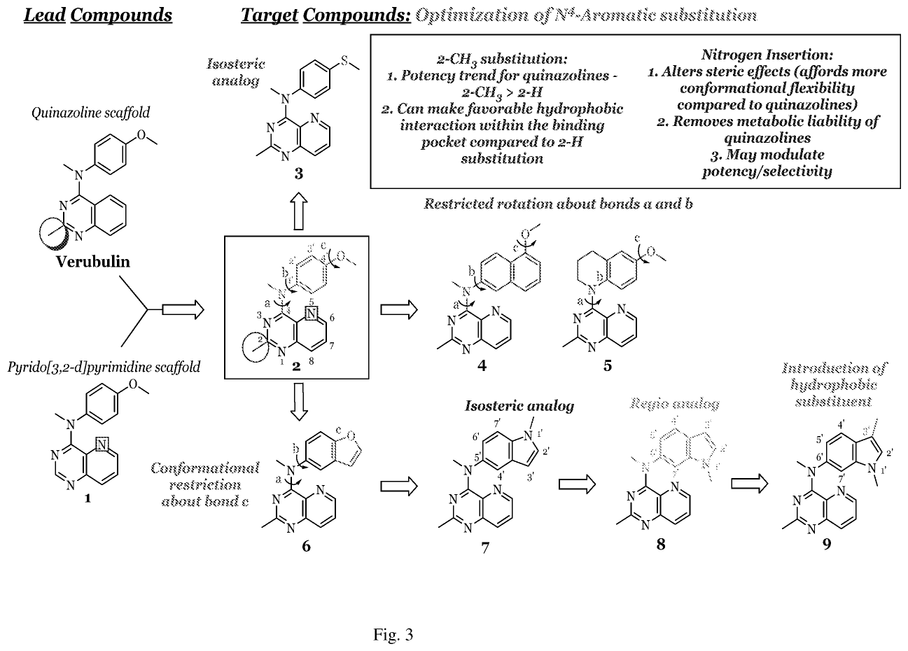

### DETAILED DESCRIPTION OF THE INVENTION

As used herein, the term “patient” means any member of the animal kingdom, including but not limited to, Homo sapiens.

As used herein, the term “effective amount” or “therapeutically effective amount” refers to that amount of any of the present compounds, salts thereof, and/or compositions required to bring about a desired effect in a patient. The desired effect will vary depending upon the illness or disease state being treated. For example, the desired effect may be reducing the tumor size, destroying cancerous cells, and/or preventing metastasis, any one of which may be the desired therapeutic response. On its most basic level, a therapeutically effective amount is that amount of a substance needed to inhibit mitosis of a cancerous cell. As used herein, “tumor” refers to an abnormal growth of cells or tissues of the malignant type, unless otherwise specifically indicated and does not include a benign type tissue. The “tumor” may be comprised of at least one cell and/or tissue. The term “inhibits or inhibiting” as used herein means reducing growth/replication. As used herein, the term “cancer” refers to any type of cancer, including but not limited to lung cancer, pancreatic cancer, and the like.

As used herein, the term “pharmaceutically acceptable vehicle” refers to a pharmaceutical vehicle or carrier that is compatible with a compound, such as for example but not limited to, normal saline (0.9% saline, physiologic saline), dextrose in water, lactated Ringer's solution, or inverted sugar solution.

As will be understood by one skilled in the art, a therapeutically effective amount of said compound can be administered by any means known in the art, including but not limited to, injection, parenterally, intravenously, intraperitoneally, orally or, where appropriate, topically. It is well within the skill of one practicing in the art to determine what dosage, and the frequency of this dosage, which will constitute a therapeutically effective amount for each individual patient, depending on the severity or progression of cancer or cancer cells and/or the type of cancer. It is also within the skill of one practicing in the art to select the most appropriate method of administering the compounds based upon the needs of each patient.

In certain embodiments of this invention, we provide a 2-methylpyrido[3,2-d]pyrimidine compound 2 of this invention using Verubulin²²⁻²³ (quinazoline scaffold) and known in the art compound 1²⁴ (pyrido[3,2-d]pyrimidine scaffold) as our leads. Compounds 3-9 of this invention are provided with different substitutions at the N4-position of compound 2 of this invention.

In certain embodiments of this invention, a compound is provided having a structure of the formula:

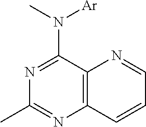

wherein Ar is a structure of the formula selected from one of the group consisting of 2, 3, 4, 6, 7, 8, and 9:

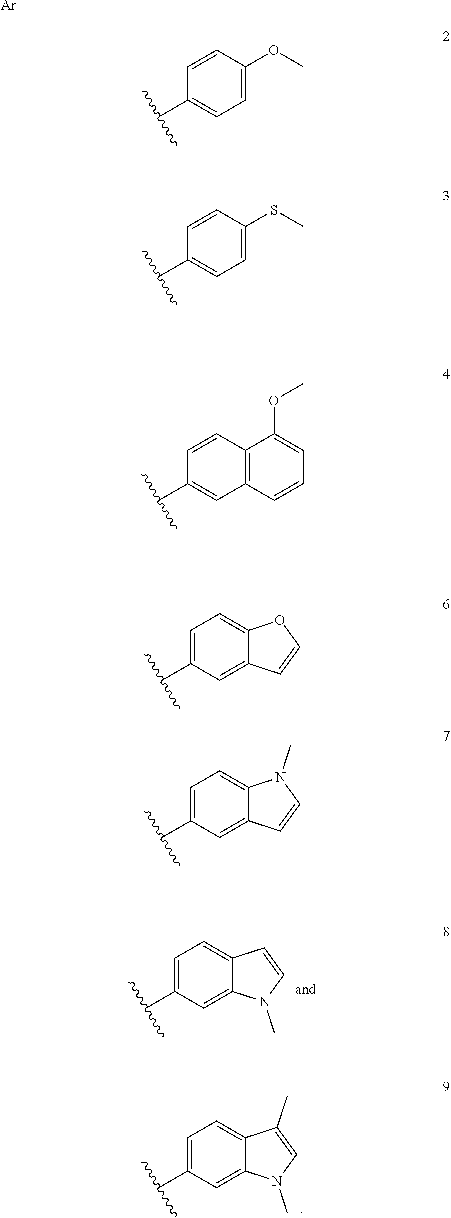

In another embodiment of this invention, a composition is provided comprising a compound having a structure of the formula:

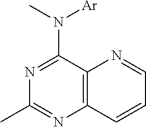

wherein Ar is a structure of the formula selected from one of the group consisting of 2, 3, 4, 6, 7, 8, and 9:

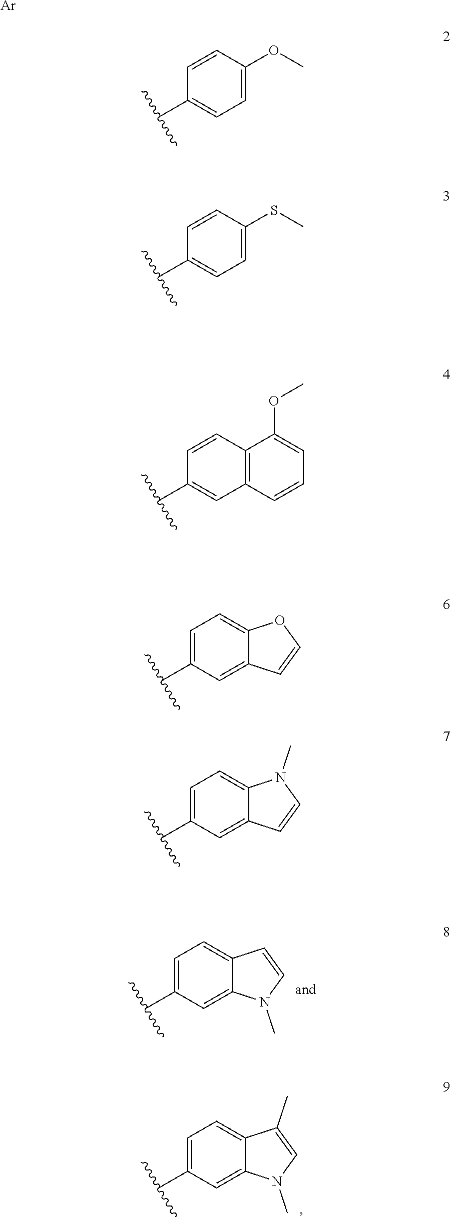

and a pharmaceutically acceptable vehicle.

In another embodiment of this invention, a method of treating a patient having cancer is provided comprising administering to said patient a therapeutically effective amount of a compound having a structure of the formula:

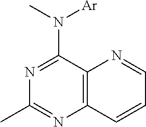

wherein Ar is a structure of the formula selected from one of the group consisting of 2, 3, 4, 6, 7, 8, and 9:

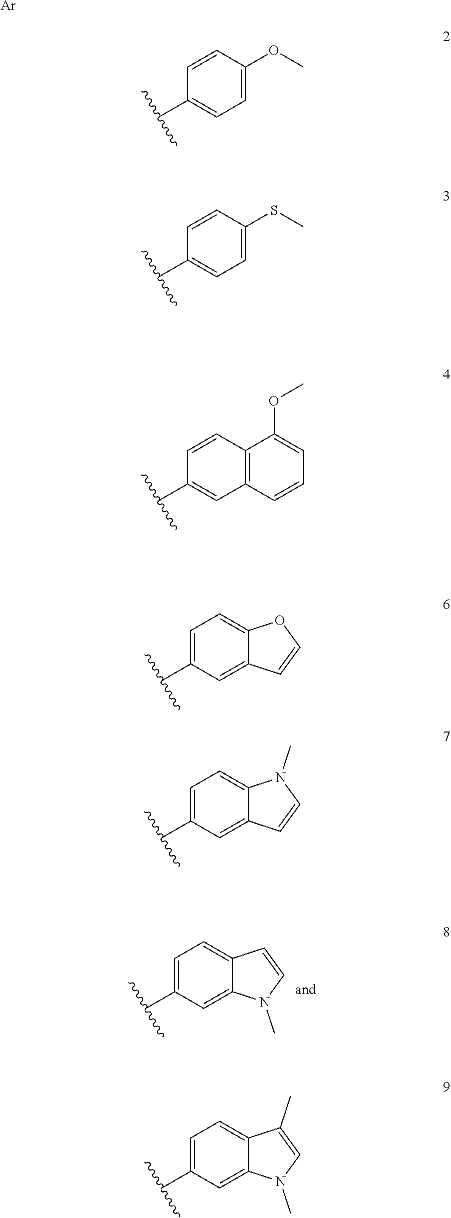

for treating said patient.

In another embodiment of this invention, a method of treating a patient having cancer is provided comprising administering to said patient a therapeutically effective amount of a composition comprising a compound having a structure of the formula:

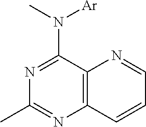

wherein Ar is a structure of the formula selected from one of the group consisting of 2, 3, 4, 6, 7, 8, and 9:

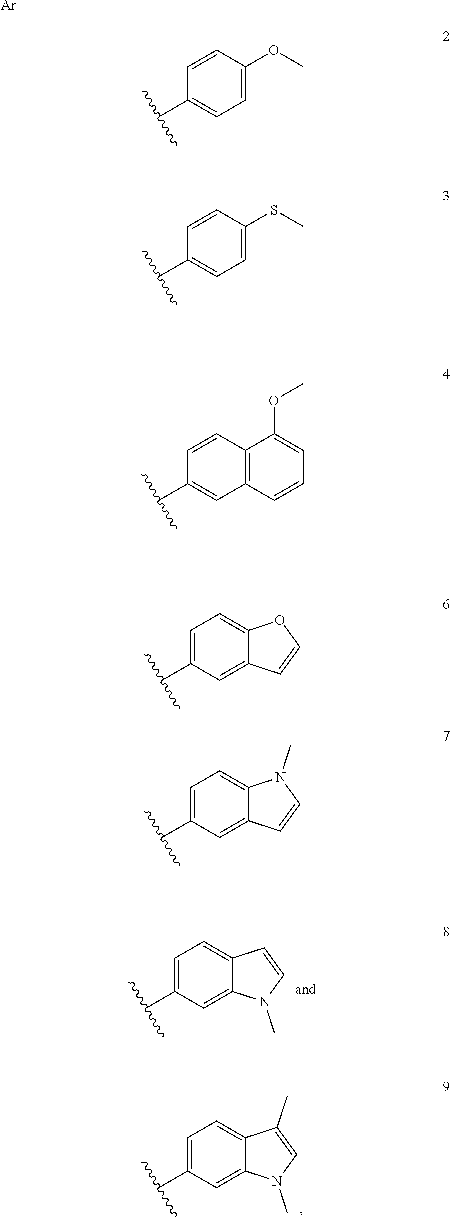

and a pharmaceutically acceptable vehicle, for treating said patient.

In another embodiment of this invention, a compound having the structure of the formula:

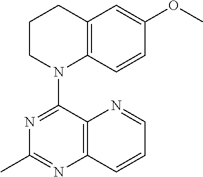

is provided.

In another embodiment of this invention, a composition is provided comprising a compound having a structure of the formula:

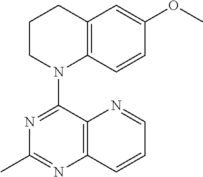

and a pharmaceutically acceptable vehicle.

Another embodiment of this invention provides a method of treating a patient having cancer comprising administering to said patient a therapeutically effective amount of a compound having a structure of the formula:

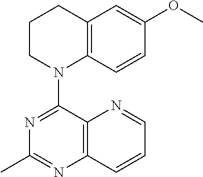

for treating said patient.

In another embodiment of this invention, a method of treating a patient having cancer is provided comprising administering to said patient a therapeutically effective amount of a composition comprising a compound having a structure of the formula:

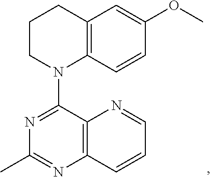

and a pharmaceutically acceptable vehicle, for treating said patient.

In the methods of the present invention of treating a patient having cancer, the cancer may be for example but not limited to breast cancer and ovarian cancer. The novel compounds and pharmaceutically acceptable salts thereof provide for treatment of tumors, or other cancer cells, in cancer patients. The types of cancer can vary widely and in certain embodiments, the novel compounds and pharmaceutically acceptable salts thereof are particularly useful for example, in treating certain breast and ovarian cancers.

TABLE 1
IC₅₀ values for inhibition of proliferation of MDA-MB-435 cells and
EC₅₀ values for microtubule depolymerization in A-10 cells.²²⁻²⁴

| | IC₅₀ ± SD in | EC₅₀ for Microtubule | |
|---|---|---|---|
| | MDA-435 Cells | Depolymerization in | EC₅₀/IC₅₀ |
| Compound | (nM) | A · 10 Cells (nM) | Ratio |
| Verubulin | 1.7 ± 0.1 | 2.1 | 1.2 |
| 1 | 30.1 ± 3 | 272 | 9.0 |
| CA4 | 4.4 ± 0.3 | 9.8 | 2.2 |

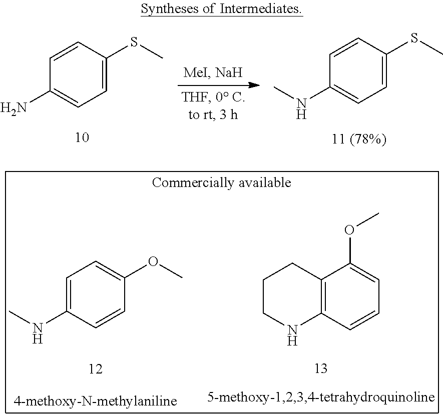

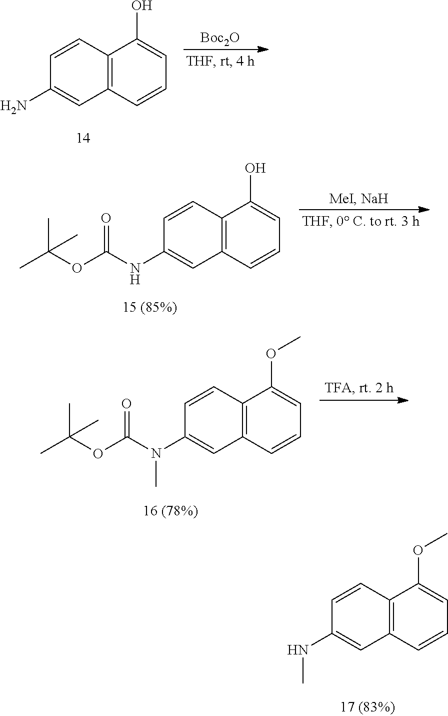

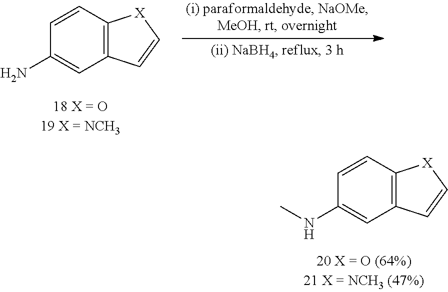

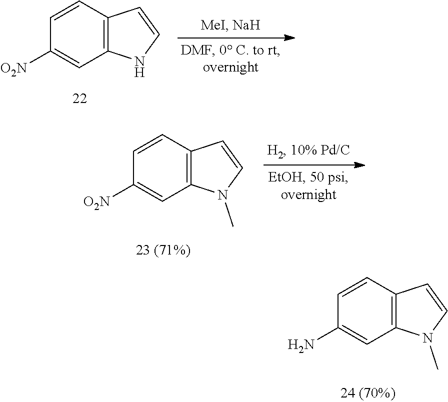

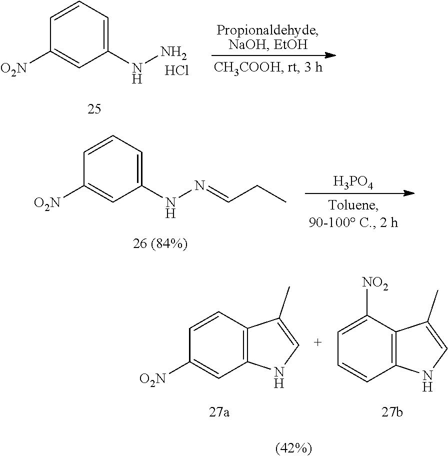

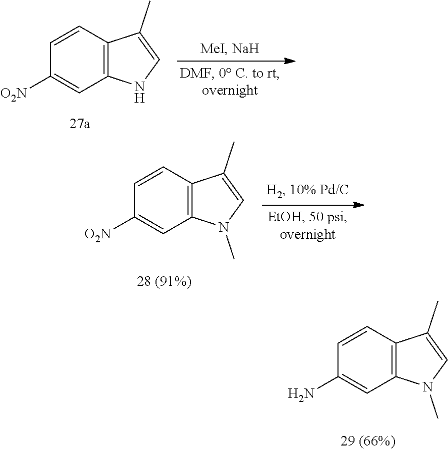

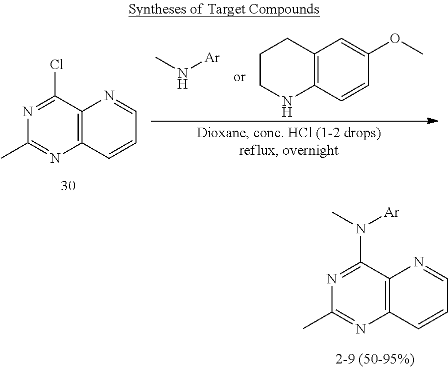

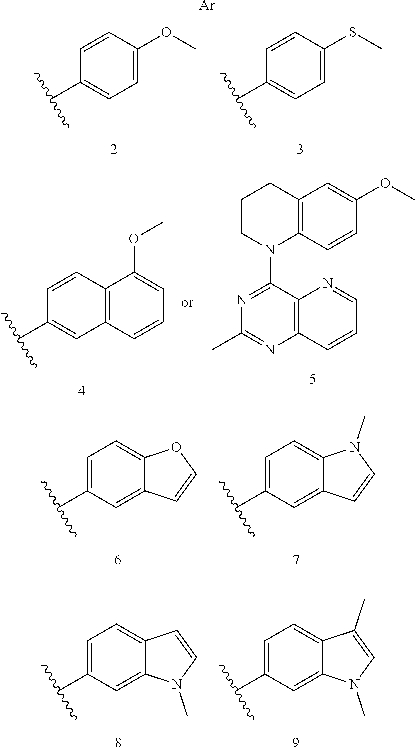

### Biological Activity:

TABLE 2
Inhibition of colchicine binding and tubulin assembly and activities
in the MCF-7, OVCAR-8, and NCI/ADR-RES cell lines.

| | | | | | Compound activity in a parental | | |
|---|---|---|---|---|---|---|---|
| | | | | | cell line (OVCAR-8) and a Pgp | | |
| | Inhibition of | | | | overexpressing cell line | | |
| | colchicine | | Inhibition | | (NCI/ADR-RES) | | |
| | binding | | of tubulin | MCF-7 | | NCI/ADR- | |
| | % inhibition ± SD | | assembly | cytotoxicity | OVCAR-8 | RES | |
| | 5 μM | 0.5 μM | IC₅₀ (μM) ± | IC₅₀ (nM) ± | IC₅₀ (nM) ± | IC₅₀ (nM) ± | |
| Compound | inhibitor | inhibitor | SD | SD | SD | SD | Rr |
| CA4 | 98 ± 0.2 | 83 ± 2 | 0.91 ± 0.1 | 9.0 ± 1 | 3.0 ± 1 | 5.0 ± 2 | 1.67 |
| Paclitaxel | — | — | — | 5.0 ± 1 | 4.0 ± 1 | 1,500 ± 70 | 375 |
| 1 | 87 ± 0.6 | ND | 1.3 ± 0.04 | ND | 68 ± 20 | 39 ± 2 | 0.57 |
| 2 | 95 ± 2 | 84 ± 1 | 1.3 ± 0.1 | 14 ± 2 | 7.5 ± 0.7 | 4.0 ± 1 | 0.53 |
| 3 | 99 ± 2 | 90 ± 2 | 0.42 ± 0.08 | 13 ± 4 | 24 ± 4 | 4.3 ± 1 | 0.18 |
| 4 | 95 ± 2 | 89 ± 0.6 | 1.3 ± 0.06 | 63 ± 20 | 5.0 ± 0 | 4.5 ± 0.7 | 0.9 |
| 5 | 98 ± 1 | 81 ± 2 | 0.36 ± 0.02 | 34 ± 5 | 11 ± 3 | 7.3 ± 3 | 0.66 |
| 6 | 85 ± 0.4 | | 0.93 ± 0.1 | 33 ± 6 | 38 ± 7 | 24 ± 7 | 0.63 |
| 7 | 97 ± 0.5 | 81 ± 2 | 0.72 ± 0.1 | 2.5 ± 0.7 | 3.0 ± 0.6 | 3.0 ± 0 | 1.00 |
| 8 | 66 ± 4 | | 1.5 ± 0.2 | 190 ± 40 | 320 ± 40 | 260 ± 30 | 0.81 |
| 9 | 93 ± 1 | 52 ± 4 | 0.89 ± 0.05 | 490 ± 40 | 25 ± 5 | 24 ± 4 | 0.96 |

ND = Not determined.

### Molecular Modeling

The colchicine binding site lies in the intermediate domain at the interface of α and β subunits. The αβ-tubulin dimer was docked. Hydrogen bonding and aromatic C—H—O interaction was observed. Superimposition of docked poses of Verubulin (docked score: −9.50 kcal/mol), lead compound 1 (docked score: −9.02 kcal/mol), and compound 2 of this invention (docked score: −8.29 kcal/mol) in the colchicine site of β-tubulin (PDB: 6BRF²¹) was observed. Superimposition of docked poses of lead compound 1 (docked score: −9.02 kcal/mol), compound 7 of this invention (docked score: −10.71 kcal/mol), and compound 8 of this invention (docked score: −10.63 kcal/mol) was observed in the colchicine site of β-tubulin (PDB: 6BRF).²⁶

### Results

Compounds 1-9 were tested to determine the direct effects on inhibition of colchicine binding and tubulin assembly. The 2-Me analog compounds 2-9 of this invention bind to the colchicine site more strongly than 2-H analog compound 1. This correlates well with the docking scores for compounds 2-9 (docking scores ranging from −8.29 to −10.71 kcal/mol) compared to compound 1 (docking score −9.02 kcal/mol). Compound 3 (0.42 μM), compound 5 (0.36 μM), and compound 7 (0.72 μM) inhibit tubulin assembly as potently as CA4 (0.91 μM).

Compound 7, with the bicyclic 1-methyl-1H-indol-5-yl substitution at N4, showed the best activity against the MCF-7 breast cancer cell line, with an improvement in cytotoxicity by 3.6- and 2-fold over CA4 and paclitaxel, respectively.

Remarkably, all the designed compounds 2-9 of this invention were found to be more potent in the Pgp overexpressing cell line (NCI/ADR-RES) compared to the parental line (OVCAR-8), whereas paclitaxel was completely inactive (IC₅₀=1,500 nM) in the Pgp overexpressing cell line. Compound 3 of this invention with a N-methyl-4-(methylthio)aniline substitution showed a resistance ratio (Rr) of 0.18 compared to 1.67 and 375 with CA4 and paclitaxel, respectively. This suggests that compounds 2-9 of this invention are poor substrates for transport by Pgp, indicating a potential utility against paclitaxel resistant ovarian cancer.

Compound 7 inhibited the growth of human ovarian cancer cell line (OVCAR-8) at IC₅₀ '₂ 3.0 nM and was comparable with CA4 (IC₅₀=3.0 nM) and paclitaxel (IC₅₀=4.0 nM). For Pgp overexpressing cell line (NCI/ADR-RES), compound 7 of this invention showed the most potent inhibition (IC₅₀ '₂ 3.0 nM).

Those persons of ordinary skill in the art will appreciate that this invention provides certain colchicine site binding pyrido[3,2-d]pyrimidine analogs with antiproliferative activity against the resistant Pgp overexpressing ovarian cancer cell line NCI/ADR-RES. Compounds 2-9 of this invention having different N4-substitutions and a bulky substitution at the 2-position, for example a methyl group, bind more strongly to the colchicine site than the desmethyl analog compound 1. Compound 3 showed a resistance ratio (Rr) of 0.18 in contrast to 1.67 for CA4 and 375 for paclitaxel, indicating its ability to circumvent Pgp-mediated resistance. Compound 7 showed the best potency against the MCF-7 breast cancer cell line as well as against both the parental (IC₅₀=3.0 nM) and Pgp overexpressing resistant (IC₅₀=3.0 nM) ovarian cancer cell lines. In addition, this study also proves that the pyrido[3,2-d]pyrimidine scaffold is as active as the quinazoline scaffold of verubulin. The presented CSBIs have potential as cancer chemotherapeutic agents owing to their potent activity against a Pgp-overexpressing resistant cancer cell line where current MTAs, including paclitaxel, fail.

In certain embodiments of the invention, the novel compounds 2-9, as described herein, include pharmaceutically acceptable salts of these compounds, and include for example but not limited to, hydrochloride (HCl) salts (or other acids) of these compounds.

It is especially advantageous to formulate parenteral compositions in dosage unit form for ease of administration and uniformity of dosage. Dosage unit form as used herein refers to physically discrete units suited as unitary dosages for the patients being treated, each unit containing a predetermined quantity or effective amount of a compound of the present invention to produce the desired effect in association with a pharmaceutical carrier. The specification for the dosage unit forms of the invention are dictated by and directly dependent on the particular compound and the particular effect, or therapeutic response, that is desired to be achieved.

Compounds 2-9, or pharmaceutically acceptable salts, or hydrates thereof, can be administered to a patient (an animal or human) via various routes including parenterally, orally or intraperitoneally. Parenteral administration includes the following routes that are outside the alimentary canal (digestive tract): intravenous; intramuscular; interstitial, intraarterial; subcutaneous; intraocular; intracranial; intraventricular; intrasynovial; transepithelial, including transdermal, pulmonary via inhalation, ophthalmic, sublingual and buccal; topical, including dermal, ocular, rectal, or nasal inhalation via insufflation or nebulization. Specific modes of administration shall depend on the indication. The selection of the specific route of administration and the dose regimen is to be adjusted or titrated by the clinician according to methods known to the clinician in order to obtain the optimal clinical response. The amount of compound to be administered is that amount which is therapeutically effective. The dosage to be administered to a patient shall depend on the characteristics of the patient being treated, including for example, but not limited to, the patient's age, weight, health, and types and frequency of concurrent treatment, if any, of any other chemotherapeutic agent(s), all of which is determined by the clinician as one skilled in the art.

Compounds 2-9, or a pharmaceutically acceptable salt, or hydrate thereof, that are orally administered can be enclosed in hard or soft shell gelatin capsules, or compressed into tablets. Compounds also can be incorporated with an excipient and used in the form of ingestible tablets, buccal tablets, troches, capsules, sachets, lozenges, elixirs, suspensions, syrups, wafers and the like. Compounds 2-9 can be in the form of a powder or granule, a solution or suspension in an aqueous liquid or non-aqueous liquid, or in an oil-in-water emulsion.

The tablets, troches, pills, capsules and the like also can contain, for example, a binder, such as gum tragacanth, acacia, corn starch; gelating excipients, such as dicalcium phosphate; a disintegrating agent, such as corn starch, potato starch, alginic acid and the like; a lubricant, such as magnesium stearate; a sweetening agent, such as sucrose, lactose or saccharin; or a flavoring agent. When the dosage unit form is a capsule, it can contain, in addition to the materials described above, a liquid carrier. Various other materials can be present as coatings or to otherwise modify the physical form of the dosage unit. For example, tablets, pills, or capsules can be coated with shellac, sugar or both. A syrup or elixir can contain the active compound, and sucrose as a sweetening agent, methyl and propylparabens as preservatives, a dye and flavoring, for example. Any material used in preparing any dosage unit form should be pharmaceutically pure and substantially non-toxic. Additionally, the compounds 2-9 of this invention, or a pharmaceutically acceptable salt, or hydrate of the compounds 2-9, can be incorporated into sustained-release preparations and formulations.

The compounds 2-9 of this invention or a pharmaceutically acceptable salt, or hydrate thereof, can be administered to the central nervous system, parenterally or intraperitoneally. Solutions of the compound as a free base or a pharmaceutically acceptable salt can be prepared in water mixed with a suitable surfactant, such as for example, but not limited to, hydroxypropylcellulose. Dispersions also can be prepared in glycerol, liquid polyethylene glycols and mixtures thereof, and in oils. Under ordinary conditions of storage and use, these preparations can contain a preservative and/or antioxidants to prevent the growth of microorganisms or chemical degeneration.

The pharmaceutical forms suitable for injectable use include, without limitation, sterile aqueous solutions or dispersions and sterile powders for the extemporaneous preparation of sterile injectable solutions or dispersions. In all cases, the form must be sterile and must be fluid to the extent that easy syringability exists. It can be stable under the conditions of manufacture and storage and must be preserved against the contaminating action of microorganisms, such as bacteria and fungi.

Compounds of the present invention may be contained within, mixed with, or associated with, a suitable (acceptable) pharmaceutical carrier for administration to a patient according to the particular route of administration desired. Suitable or acceptable pharmaceutical carriers refer to any pharmaceutical carrier that will solubilize the compounds of the present invention and that will not give rise to incompatability problems, and includes any and all solvents, dispersion media, coatings, antibacterial and antifungal agents, isotonic agents, absorption delaying agents, and the like. The use of such suitable or acceptable pharmaceutical carriers is well known by those skilled in the art. Preferred carriers include sterile water, physiologic saline, and five percent dextrose in water. Examples of other suitable or acceptable pharmaceutical carriers include, but are not limited to, ethanol, polyol (such as propylene glycol and liquid polyethylene glycol), suitable mixtures thereof, or vegetable oils. The proper fluidity can be maintained, for example, by the use of a coating, such as lecithin, by the maintenance of the required particle size (in the case of a dispersion) and by the use of surfactants. The prevention of the action of microorganisms can be brought about by various antibacterial and anti-fungal agents, for example, parabens, chlorobutanol, phenol, sorbic acid, thimerosal, and the like. In many cases, it will be preferable to include isotonic agents, for example, sugars or sodium chloride.

Sterile injectable solutions are prepared by incorporating a compound 2, 3, 4, 5, 6,7, 8 or 9 of this invention in the required amount in the appropriate solvent with various of the other ingredients enumerated above, as required, followed by filtered sterilization. Generally, dispersions are prepared by incorporating the sterilized compound 2, 3, 4, 5, 6, 7, 8 or 9, into a sterile vehicle that contains the basic dispersion medium and any of the other ingredients from those enumerated above. In the case of sterile powders for the preparation of sterile injectable solutions, the preferred methods of preparation are vacuum drying and freeze drying.

The therapeutic compounds 2, 3, 4, 5, 6,7, 8, or 9 of this invention, as described herein, may be administered to a patient alone or in combination with pharmaceutically acceptable carriers or as pharmaceutically acceptable salts, or hydrates thereof, the proportion of which is determined by the solubility and chemical nature of the compound, chosen route of administration to the patient and standard pharmaceutical practice.

The present invention is more particularly described above in the non-limiting examples, which are intended to be illustrative only, as numerous modifications and variations therein will be apparent to those skilled in the art.

It will be appreciated by those skilled in the art that changes could be made to the embodiments described above without departing from the broad inventive concept thereof. It is understood, therefore, that this invention is not limited to the particular embodiments disclosed, but it is intended to cover modifications that are within the spirit and scope of the invention, as defined by the appended claims.

What is claimed is:

1. A compound having a structure of the formula: 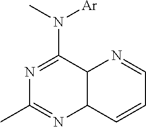
wherein Ar is a structure of the formula selected from one of the group consisting of 2, 3, 4, 6, 7, 8, and 9:

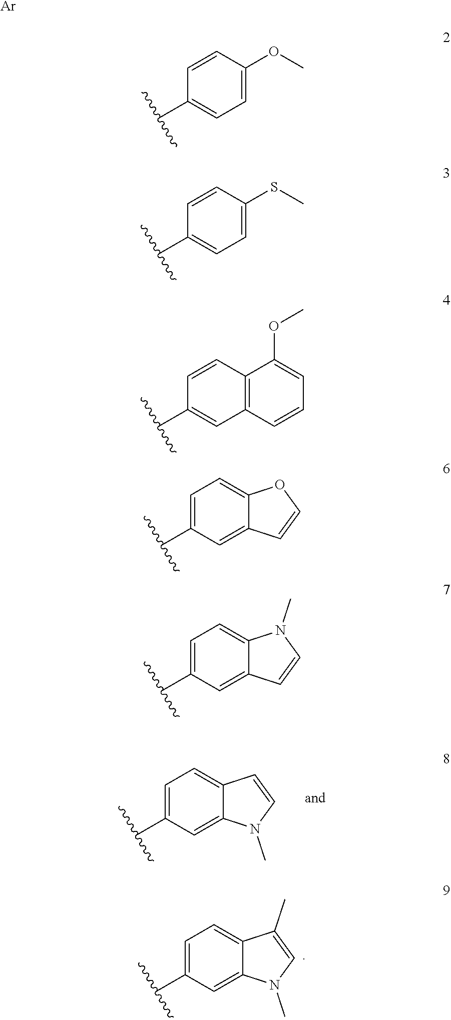

2. A composition comprising a compound having a structure of the formula: 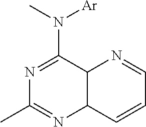
wherein Ar is a structure of the formula selected from one of the group consisting of 2, 3, 4, 6, 7, 8, and 9: 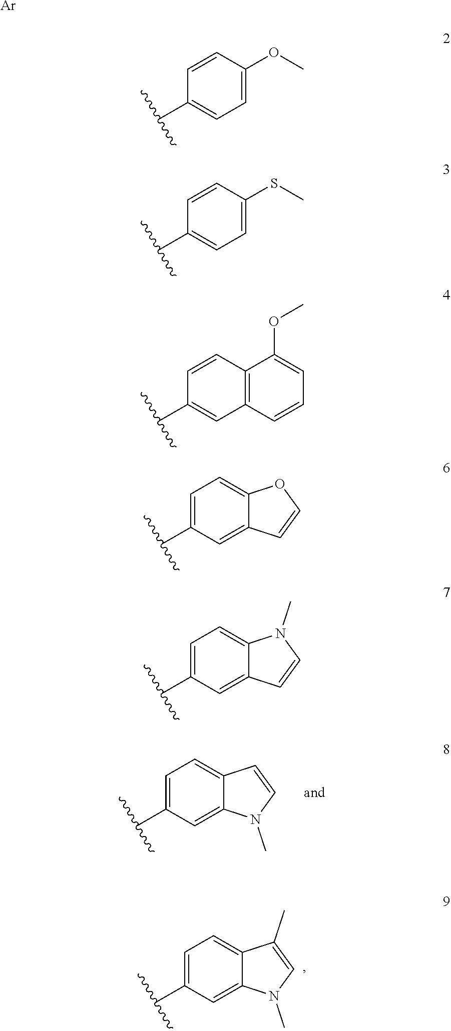
and a pharmaceutically acceptable vehicle.

3. A method of treating a patient having cancer comprising administering to said patient a therapeutically effective amount of a compound having a structure of the formula: 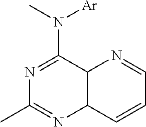
wherein Ar is a structure of the formula selected from one of the group consisting of 2, 3, 4, 6, 7, 8, and 9: 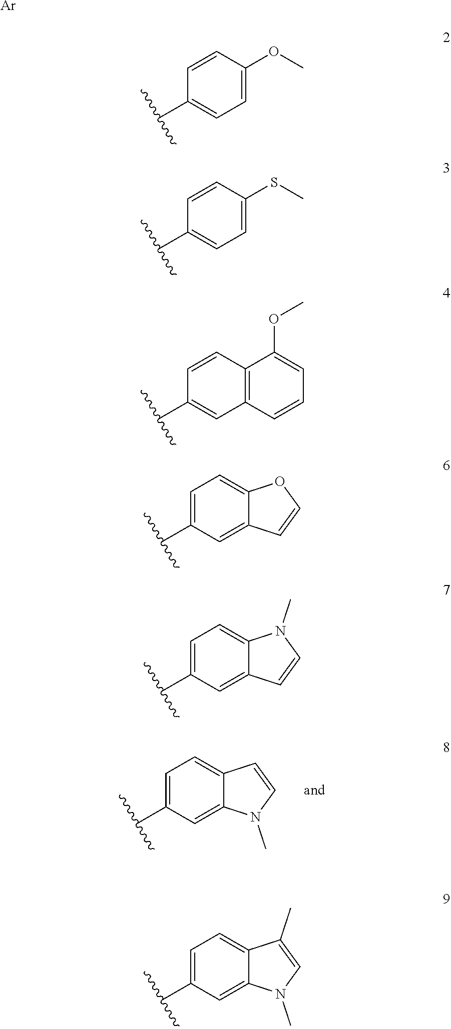
for treating said patient.

4. A method of treating a patient having cancer comprising administering to said patient a therapeutically effective amount of a composition comprising a compound having a structure of the formula: 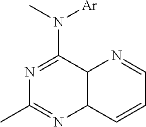
wherein Ar is a structure of the formula selected from one of the group consisting of 2, 3, 4, 6, 7, 8, and 9: 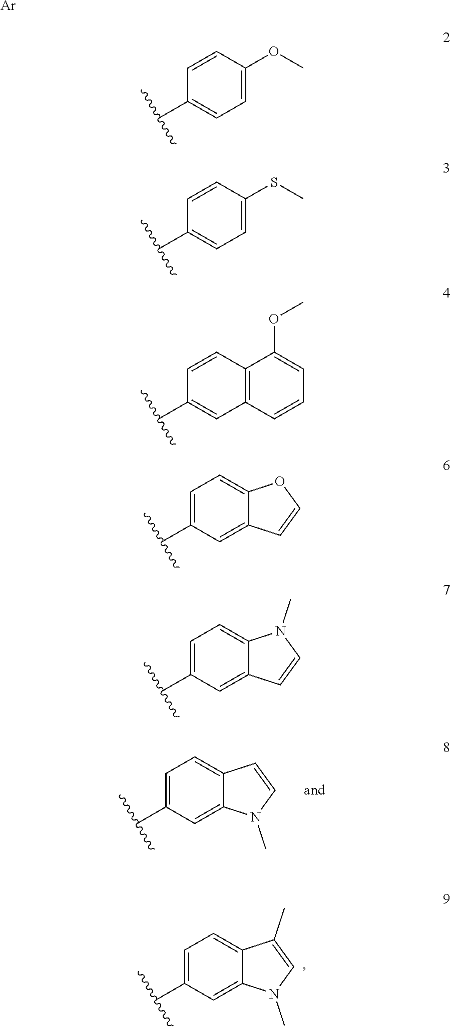
and a pharmaceutically acceptable vehicle, for treating said patient.

5. A compound having the structure of the formula:

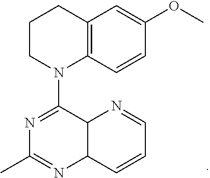

6. A composition comprising a compound having a structure of the formula: 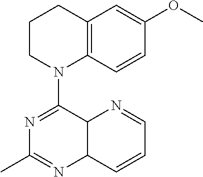
and a pharmaceutically acceptable vehicle.

7. A method of treating a patient having cancer comprising administering to said patient a therapeutically effective amount of a compound having a structure of the formula: 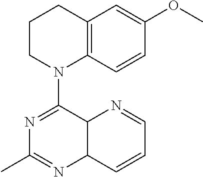
for treating said patient.

8. A method of treating a patient having cancer comprising administering to said patient a therapeutically effective amount of a composition comprising a compound having a structure of the formula: 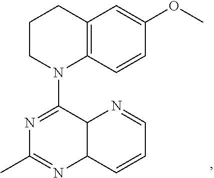
and a pharmaceutically acceptable vehicle, for treating said patient.
